(19) United States
(12) Patent Application Publication
(10) Pub. No.: US 2018/0015072 A1
(43) Pub. Date: Jan. 18, 2018

(54) Tryptophan is a pro-drug for infectious diseases and cancers

(71) Applicants: Zhifang Zhu, New York, NY (US); Jun Zhang, Shanghai (CN); Jinming Li, Shanghai (CN)

(72) Inventors: Zhifang Zhu, New York, NY (US); Jun Zhang, Shanghai (CN); Jinming Li, Shanghai (CN)

(73) Assignees: Zhifang Zhu, New York, NY (US); Jinming Li, Shanghai (CN)

(21) Appl. No.: 15/208,586

(22) Filed: Jul. 12, 2016

(51) Int. Cl. A61K 31/405; A61K 9/00; A61K 31/7056

(52) U.S. Cl. CPC A61K 31/405; A61K 31/7056; A61K 9/0014; A61K 9/0019; A61K 9/0053

(57) ABSTRACT

The innovation provides compositions and methods for treating infectious diseases and cancers, wherein said, in vitro, in higher active TDO (tryptophan 2,3-dioxygenase) sick cells that include cancer and infectious cells, tryptophan is capable of transferring into kynurenine derives to induce cells apoptosis; in vivo, tryptophan will turn into indican. Indican is capable of competing phosphorylation in the subject, competitively inhibiting process to form GTP, ATP, dGTP, dATP, that are essential for DNA/RNA synthesis; inhibiting host or invading gene amplification, or interfere with PLK(polo like kinase) phosphorylation to trigger cell apoptosis. All these function can be used for clinic treatment for cancer and infectious diseases.

### BACKGROUND OF THE INNOVATION

Tryptophan possesses variety of functions that include transferring into serotonin, kynurenine, co enzyme nicotinamide adenine dinucleotide. Under gut bacterial tryptophanase, tryptophan will turn into indole, and then transfer into indicant in liver. No any literature had revealed indican's bio-function before.

Indican possesses similar chemical structure as guanosine and adenosine, also share similar chemical reactions that include glycolization, phosphorylation. Since the free energy to form phosphorylated indicant is lower than to form ATP (1), in the same reaction pool, indicant will show great compete power to inhibit forming ATP, GTP, dATP, dGTP that are essential for gene amplification. When the pathogen gene stopping amplification, host cell DNases/Rnase will digest these pathogen genes as DNA/RNA molecules.

In all of cancer cells, PLK (polo like kinase) show high activities. Interfering PLK phosphorylation will trigger cancer cell apoptosis (2). According to chemical function, Indican interferes with PLK phosphorylation too.

In cancer and infectious cells, activities of TDO and IDO significantly increased, also increased tryptophan requirement (3). Cachexia, extremely poor protein degradation, is a poor predicator of cancer and chronically infectious patients (4). This innovation connected this phenomenon as biological individual self-protection. As a rate control factor, tryptophan senses the body's requirement. If there is no enough tryptophan in blood system, body has to degrade structure proteins to generate tryptophan. The innovation reveals that Cachexia is self-protection to produce tryptophan to fight nun-selves (pathogens.) Relay on the fact that kynurenine can induce apoptosis (5). Higher TDO and IDO in sick cells (cancer and infectious cells) offer specificity for tryptophan to target them. This concept is part of foundation for this innovation, but is against certain research group that they think tryptophan enhance cancer genesis (5)

Tryptophan is an essential amino acid. It is synthesized in prokaryote system. The related gene expression is regulated by negative feedback regulation style (6). In human, most of tryptophan molecules are combined with albumins (7). These two independent phenomenons reveal that its concentration has to be controlled restrictively. The biological significance could be:
1. To avoid toxicity to the system;
2. To maintain its rate controller in protein synthesis and degradation. Among 22 amino acids, tryptophan maintains the lowest concentration in human that acts protein metabolism process.

This significance related to innate immune-response. The deficiency of tryptophan will cause innate response improper. It is the foundation for prevention.

Laboratory Data

In vitro:
1. Tryptophan (named ZZ) and indicant (named LL); both inhibit randomly selected pancreatic cancer Mia PaCa2 cell growth. (tested in South Research Ins. Reported by Koratich Mike on 09-24-2015)
2. Both of Tryptophan and Indican inhibit Herpes simplex virus 1; vaccinia virus; human cytomegalovirus; poliovirus; rift valley fever virus; tacaribe virus. (By NIH anti-virus primary screen test as ARB 16-000227 (tryptophan), ARB 16-000226 (indicant) reported by USB, SRI, and USU labs.)
3. Among 15 groups of virus tests by both China and USA, Indican inhibits 14 groups of viruses that include yellow fever virus (as sample B, reported by China Shanghai Pastern Ins.); inhibits chikungunya viruses; dengue viruses; influenza A H1N1 viruses; MERS coronavirus; polio viruses 3; respiratory syncytial viruses; Rift valley fever viruses; Tacaribe viruses; Venezuelan equine encephalitis viruses; hepatitis C virus. (By NIH anti-virus primary screen test as ARB 16-000226 (indican) reported by USB, SRI, and USU labs.) The only one does not show function is hepatitis B that is conflict to Chinese clinical experience. [Indican is rich in ban Ian gen(Isatis), ban Ian gen extracts is selected by CFDA for HBV treatment]. The data indicate that Indican inhibits all of viruses as our conclusion.
4. Under 0.5 mg/ml, Tryptophan is safe for human primary white cells (reported by south research Ins. Project #14834.01)

In vivo:

At 80 mg/kg/day dosage, tryptophan and indicant both show significant prevention and early stage treatment function in nun small cell lung cancer mice model. (As sample A reported by China Tongi medical university)

At 320 mg/kg/day dosage, tryptophan and indicant both show significant function in hand foot mouth disease mice model. (Reported by China food and drug inspection Ins.)

### SUMMARY

The innovation is based on summarizing from clinical experience, logical conclusion, then doing experiment again. Limited by conditions, we are not able to test every virus and cancer at this moment, but the selected cancer cell line and mice model are based on reality that is random selection. From these random selection, both tryptophan and indicant show significant function.

As we indicated drug, indicant shows universal anti-virus function in setting tests. That approved our logical conclusion. Tryptophan has to be transfer into indican through metabolism system, so it is not strange that it can show partial function in vitro from laboratory data. Its universal function has to be detected by further animal tests.

As an endogenous molecule, tryptophan related to basic protein structure, hormones-enzyme and neuro transmitter synthesis. Through its metabolism product Indican we made conclusion and approved by tests that tryptophan also possesses function for anti-cancer and anti-infectious diseases. Along with other amino acids, lysine also shows anti-virus function that revealed an interesting phenomenon that any existed

Tryptophan is a pro drug for infectious diseases and cancers individual possesses huge potential power to live. Instead of searching/designing powerful also toxic drugs, understanding/finding these unknown endogenous molecules' function will offer better benefit to our lives. They may not as powerful as man-made/computer made molecules, but they must maintain constant functions. Prediction is that medicine finally will go back to its original-self healings, since it is a safer path. Comparing with current existed or are processing anti-cancer/infectious drugs, tryptophan possesses following advantages:
1. Safer, the biological system can automatically regulate its free molecules concentration.
2. Avoiding MDR (multiple drug resistant) that is nightmare for all of chemo drugs.
3. Avoiding selection pressure caused drug abuse, to stop inducing super pathogens.
4. Gene level controlled work principle plus innate immune regulation, it can fight any mutated pathogens.
5. Variety of administration offers great convenience for clinic.
6. To avoid apoptosis caused side effects, cooperating with other factors are considered.

### REFERENCE

1. Chemistry 423 lecture 15 application of free energy
2. Lowery M D et al. Structure and function of polo like kinase. Oncogene. 2005 Jan. 10; 24 (2):248-59
3. Nesrine Kamal et al. Metabolism indoleamine 2,3-dioxygenase friend and foe. J. Metabonomics 1:2 doi: 10.417/2325-9736
4. Corie Lok, Cachexia, The last illness, Nature 9 Dec. 2015
5. Platten et al. Tryptophan catabolism in cancer. Cancer Research 2012 No 172(21):5435
6. C. Yanotsky and Horn, Role of regulatory features of Tro operon. J. Bacterol 1994 Octpber 176(20):6245-54
7. S. Comai, et al Serum levels of Tryptophan, J Tryptophan Res. 2010; 3:69-75

1. The claim covers all of the treatment and prevention for infectious diseases and cancers by Tryptophan and indican that is Tryptophan metabolism product.

2. The claim covers all the treatment and prevention that include oral administration, injection, and cover or washing lessons that caused by infections.

3. The claim covers artificial modified Tryptophan and indican molecules for infectious diseases and cancers.
